(19) United States
(12) Patent Application Publication
(10) Pub. No.: US 2023/0296609 A1
(43) Pub. Date: Sep. 21, 2023

(54) CAPTURING METHYLATED NUCLEIC ACIDS

(71) Applicant: Watchmaker Genomics, Inc., Boulder, CO (US)

(72) Inventor: Julie Walker, Wheat Ridge, CO (US)

(21) Appl. No.: 18/184,874

(22) Filed: Mar. 16, 2023

Related U.S. Application Data
(60) Provisional application No. 63/321,024, filed on Mar. 17, 2022.

(51) Int. Cl. G01N 33/574; G01N 33/53; C12Q 1/6804; C12Q 1/6886

(52) U.S. Cl. CPC G01N 33/57484; G01N 33/5308; C12Q 1/6804; C12Q 1/6886; C12Q 2600/154

(57) ABSTRACT

The present invention provides methods and compositions that use a polypeptide that includes a methyl-CpG-binding domain to capture and isolate methylated DNA from a sample.

### SEQUENCE LISTING

A “Sequence Listing XML” is submitted herewith in XML file format and (i) the name of the file is WMG-010-01WO.xml; (ii) the date of creation is Mar. 16, 2023; and (iii) the size of the file is 7,481 bytes and the material in the XML file is incorporated by reference.

### TECHNICAL FIELD

The present disclosure generally relates to methods and compositions that capture methylated nucleic acids using a capture moiety comprising a methyl-CpG-binding domain.

### BACKGROUND

Studies have shown that epigenetics can play a critical role in disease. One of the more commonly studied epigenetic modifications is DNA methylation. DNA methylation is thought to help regulate gene expression. Thus, many diseases, such as cancer, have been associated with increased levels of DNA methylation. In cells, most DNA methylation occurs via the addition of a methyl group to the 5′ carbon of a cytosine residue (5 mC), particularly on cytosines found among stretches of CpG dinucleotides (CpG islands). It is thought that, in certain circumstances, increased methylation (hypermethylation) is a natural response to certain conditions (e.g., cancer), which helps reduce transcription of certain genes. In cells, methylated DNA can reduce transcription by preventing the binding of certain transcription factors and/or may recruit repressor complexes to a gene.

Due to its emerging role in cancer, there has been increased interest in assessing the methylation status of genetic material from cancer cells, e.g., tumor cells. Cell-free DNA (cfDNA) from cancer cells, such as circulating tumor DNA (ctDNA), has a distinct methylation status compared to other cfDNA. Accordingly, it represents a promising avenue for cancer diagnosis and study.

### SUMMARY

The present invention provides methods and compositions that use a polypeptide that includes a methyl-CpG-binding domain to capture and isolate methylated DNA from a sample. In preferred aspects, the captured DNA is cell-free DNA (cfDNA) such as circulating tumor DNA (ctDNA). cfDNA is an important biomarker for assessing and diagnosing cancer and is readily identified from other cfDNA due to its methylation status. Moreover, the methylation profile of ctDNA, including any changes, may be used to detect, classify, and monitor the progression of a cancer or its treatment.

Although methods for capturing and isolating methylated DNA exist, these prior methods, such as methylated immunoprecipitation coupled with next-generation sequencing in the process known as methylated DNA immunoprecipitation sequencing (MeDIP-seq), require high concentrations of DNA and/or are unable to capture and isolate short nucleic acids. However, ctDNA is generally short in length (usually less than 200 bp) and found in low concentrations. The presently disclosed methods and composition using a polypeptide that includes a methyl-CpG-binding domain (MBD) are able to capture short, methylated-DNA sequences found at low concentrations.

The naturally occurring methyl-CpG binding domain protein 2 (MBD2) has the ability to specifically bind to 5 mC, particularly when found in CpG islands. However, the present Inventors have discovered that a wide array of polypeptide methyl-CpG-binding domains, including those engineered from naturally occurring MBD2 sequences, are able to capture methylated nucleotides.

In certain aspects, the invention provides methods for assessing a nucleic acid. An exemplary method comprises providing a sample comprising a methylated nucleic acid; contacting the sample with a polypeptide comprising methyl-CpG-binding domain (MBD); and capturing the methylated nucleic acid with the polypeptide to isolate the methylated nucleic acid. In certain aspects, the methylated nucleic acid is a cell-free DNA (cfDNA). The cfDNA may be a circulating tumor DNA (ctDNA).

In certain aspects, the polypeptide comprises a plurality of MBDs. In certain aspects, the polypeptide includes or is bound to a capture tag. In certain aspects, the polypeptide is attached to a solid surface via the capture tag. In certain aspects, the polypeptide is attached to the solid surface after capturing the methylated DNA. In certain aspects, the polypeptide is attached to the solid surface before capturing the methylated DNA. In certain aspects, the capture tag is cleavable. In such methods, the polypeptide with captured methylated nucleic acid may be released from the solid surface via cleavage of the capture tag.

Exemplary capture tags include Protein A, Protein G, Streptavidin, Cell Surface Vimentin 5 (CSV), Human Prostate-specific Membrane Antigen (PSMA), antibodies, antibody fragments, IgG Fc heavy chain, a viral protein, a chitin binding domain, a maltose binding protein, Protein L ‘B’ repeats, oligonucleotides, and other capture tags known in the art. Capture tags may be captured by a cognate moiety attached to a solid surface. The solid surface may include, for example, a bead, flow cell, or column.

In certain aspects, the MBD is from or derived from MBD2.

### BRIEF DESCRIPTION OF THE DRAWINGS

FIG. 1 shows a structure of a protein referred to as “prod-aqg”.

FIG. 2 shows a structure of a protein referred to as “prod-bqi”.

FIG. 3 shows a structure of a protein referred to as “prod-arj”.

FIG. 4 is a gel showing that the protein referred to as “prod-aqg” was produced in E. coli.

FIG. 5 is a gel showing that the protein referred to as “prod-bqi” was produced in E. coli.

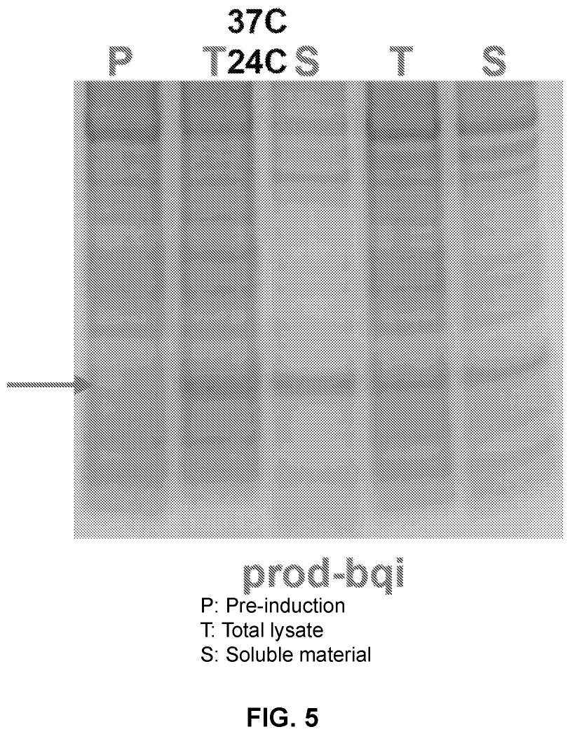

FIG. 6 is a gel showing that the protein referred to as “prod-arj” was produced in E. coli.

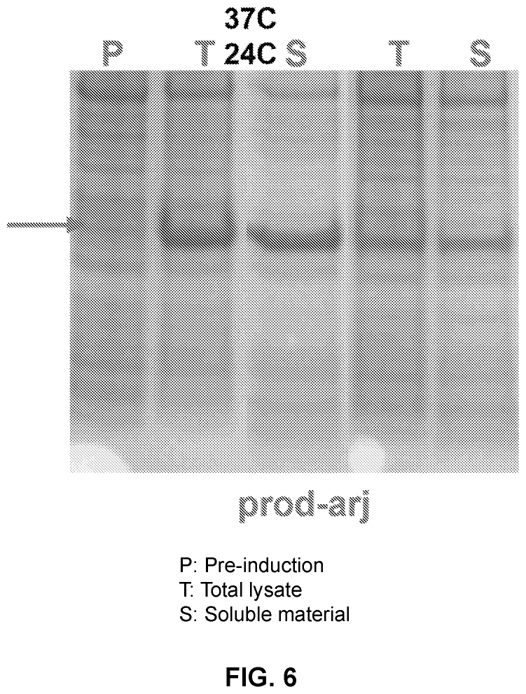

### DETAILED DESCRIPTION

The present invention provides methods and compositions that use a polypeptide that includes a methyl-CpG-binding domain to capture and isolate methylated DNA from a sample. In preferred aspects, the captured DNA is cell-free DNA (cfDNA) such as circulating tumor DNA (ctDNA). cfDNA is an important biomarker for assessing and diagnosing cancer and is readily identified from other cfDNA due to its methylation status. Moreover, the methylation profile of ctDNA, including any changes, may be used to detect, classify, and monitor the progression of a cancer or its treatment.

In certain aspects, the invention provides methods for assessing a nucleic acid. An exemplary method comprises providing a sample comprising a methylated nucleic acid; contacting the sample with a polypeptide comprising methyl-CpG-binding domain (MBD); and capturing the methylated nucleic acid with the polypeptide to isolate the methylated nucleic acid. In certain aspects, the methylated nucleic acid is a cell-free DNA (cfDNA). The cfDNA may be a circulating tumor DNA (ctDNA).

In certain aspects, the polypeptide comprises a plurality of BMDs. The plurality of BMDs may be used to capture a plurality of methylated nucleic acids simultaneously and/or capture a plurality of methylated based in a single methylated nucleic acid.

The naturally occurring methyl-CpG binding domain protein 2 (MBD2) has the ability to specifically bind to 5 mC, particularly when found in CpG islands. However, the present Inventors have discovered that a wide array of polypeptide methyl-CpG-binding domains, including those engineered from naturally occurring MBD2 sequences, are able to capture methylated nucleotides.

In certain aspects, the polypeptide includes or is bound to a capture tag. In certain aspects, the polypeptide is attached to a solid surface via the capture tag. In certain aspects, the polypeptide is attached to the solid surface after capturing the methylated DNA. In certain aspects, the polypeptide is attached to the solid surface before capturing the methylated DNA. In certain aspects, the capture tag is cleavable. In such methods, the polypeptide with captured methylated nucleic acid may be released from the solid surface via cleavage of the capture tag.

Table 1 below provides exemplary capture tags and cognate ligands attached to exemplary types of solid supports.

TABLE 1

| Capture tag | Cognate ligand & surface | Additional Comments |
|---|---|---|
| His Tag | Nickel beads/surface | Nickel-6xHis is stable |
| | | in 2M salt |
| GST | glutathione | |
| Protein A | IgG-conjugated beads, | |
| | Ka 8.02 × 10\hat{ }3 M-1 s-1 | |
| Protein G | IgG-conjugated beads, | |
| | Ka 3.29 × 10\hat{ }4 M-1 s-1 | |
| Streptavidin | Biotin beads | Streptavidin tetramerizes-can |
| | | engineer momomeric form. |
| Cell Surface Vimentin (CSV) | CSV monoclonal Ab beads | CSV may |
| | | dimer/tetramer/oligomerize |
| Human PSMA | PMSA monoclonal Ab beads | |
| IgG Fc heavy chain | Protein A/G beads | Protein A/G binding location |
| COVID-19 spike protein | Human ACE-2 conjugated beads | |
| Chitin binding domain | Chitin beads | |
| Maltose binding protein | Amylose or anti-MBP beads | |
| (MBP) | | |
| Protein L ‘B’ repeats | IgG-conjugated beads | B-repeats responsible |
| | | for Ab binding |
| SpyTag | SpyCatcher | |

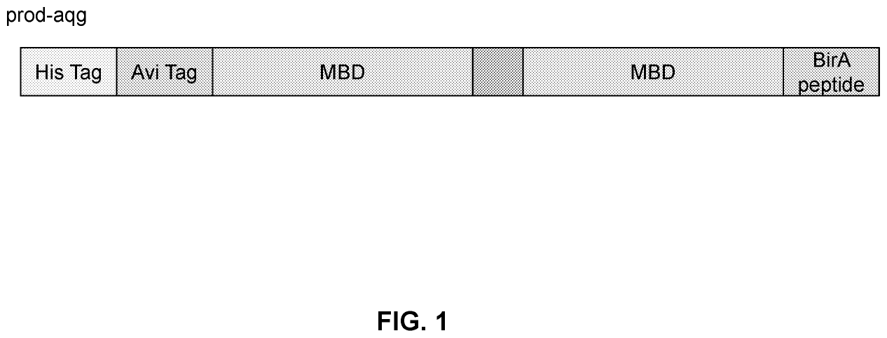

FIGS. 1-3 provide schematics of exemplary polypeptides of the invention.

FIG. 1 shows a schematic of exemplary polypeptide “prod-aqg” of the invention. The polypeptide is a fusion protein that includes an MBD. The MBD may be from or derived from Methyl-CpG Binding Domain Protein 2 (MBD2). However, the present Inventors have discovered that MBD of different polypeptide sequences readily capture methylated nucleic acids. The fusion protein also includes a capture tag, in this case, a poly-histidine (His Tag), which may be used to attach the fusion protein to a solid surface, e.g., a bead, column, or flow cell. The His tag may be used for attachment, for example to a functionalized or nickel surface. The polypeptide may be attached to a solid surface via the tag prior to capturing the methylated nucleic acid. Alternatively, the polypeptide may capture the methylated nucleic acid and then be attached to a solid surface via the tag to isolate the capture product.

As also shown in FIG. 1, the capture tag may be attached to the polypeptide/fusion protein via a cleavable moiety or linker, in this case, an Avi Tag, which may release the polypeptide-methylated nucleic acid from the solid surface. Further, the polypeptide/fusion protein may further include an additional capture tag or other functional moiety.

As also shown in FIG. 1, the polypeptide/fusion protein may further include an enzyme, such as a ligase, polymerase, transferase, and the like. As shown in FIG. 1, the enzyme is the ligase BirA. Advantageously, BirA (bifunctional biotin-[acetylCoA carboxylase] holoenzyme synthetase/DNA-binding transcriptional repressor, bio-5′-AMP-binding) acts as both a ligase and a transcriptional repressor of the biotin operon. The biotin may be used, for example, to capture the polypeptide-methylated nucleic acid via a biotin-streptavidin interaction.

In an exemplary method, prod-aqg is introduced with Ni₂ beads and bound via the His Tag to the beads. The beads are then introduced to a sample of sheared DNA and incubated. After incubation, the beads are washed, preferably in a low salt wash. The bead-bound MBDs are eluted in a salt-based elution.

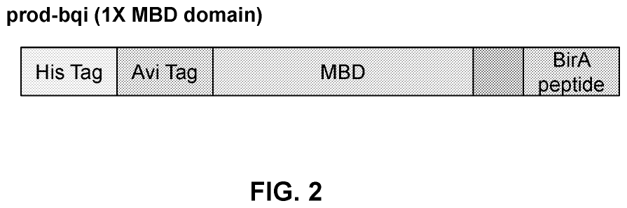

FIG. 2 provides a schematic of exemplary polypeptide “prod-bqi” of the invention. In contrast to the “prod-aqg” polypeptide, this polypeptide includes only a single MBD.

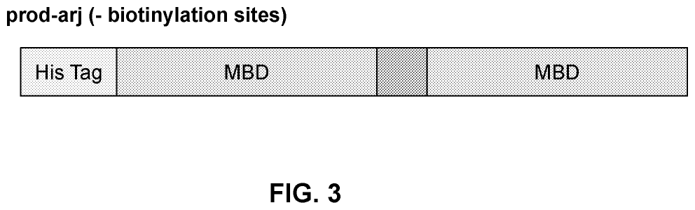

FIG. 3 provides a schematic of exemplary polypeptide “prod-arj” of the invention.

Exemplary capture tags include Protein A, Protein G, Streptavidin, Cell Surface Vimentin (CSV), Human Prostate-specific Membrane Antigen (PSMA), anbodies, antibody fragments, IgG Fc heavy chain, a viral protein, a chitin binding domain, a maltose binding protein, Protein L ‘B’ repeats, oligonucleotides, and other capture tags known in the art. Capture tags may be captured by a cognate moiety attached to a solid surface. The solid surface may include, for example, a bead, flow cell, or column.

In certain aspects, the MBD is from or derived from MBD2.

In certain aspects, the captured methylated nucleic acid is sequenced or used in another downstream application. In certain aspects, the methylation status of the nucleic acid is assessed. In certain aspects, the captured methylated nucleic acids are ctDNA. In certain aspects, the methods of the invention are used to diagnose or prognose cancer.

### EXAMPLES

### Example 1

MBD is a well characterized domain of many cytosine specific methyltransferase proteins. In this example, an MBD(s) are with other entities, which may be swiftly ‘pulled down’, i.e., MBD2-6×His can be pulled down by Nickel beads. This allows for MBD-dependent enrichment of 5 mc containing oligos from a sheared DNA sample pre-library 30 preparation.

Transformation of 1 uL 100 ng/uL pD441 expression plasmids encoding for polypeptide prod-aqg, prod-bqi, or prod-arj into 30 uL NEBExpress cells with 100 uL 20 min recovery onto LB/Kan. L LB/Kan starter cultures were started from single colonies and grown overnight at 30° C.

Starter cultures (OD600s below) were used to inoculate 1:100 single 55 mL TB/Kan culture. 100 uL starter culture was mixed with 400 uL 50% glycerol and saved at −80° C. for a working cell stock. Cultures were grown 37° C. 250 rpm until an OD600 of 1.0 (˜3 hours). At OD600 1.0, cultures were then split 2×25 mL, brought to the appropriate temperatures (37° C. vs 24° C.), and induced at 1 mM IPTG. 1 mL pre-IPTG samples were taken before induction, and pelleted.

Expression cultures were grown a further 5 hours at indicated temperature. 1 mL of culture was pelleted for soluble protein expression 20k×g 5 mins 4° C. 16° C. expression cultures were grown overnight. All cultures were grown with 250 rpm shaking in 250 m Lerlenmeyer flasks. Final 16C OD600s: 1 mL of culture was pelleted for soluble protein expression 20k×g 5 mins 4° C.

Pellets are lysed to obtain the above to analyze soluble protein expression by SDS page, which included Resuspending pellets in (0D600*100) uL of 400 mM lysis buffer, saving 50 uL ‘total’ sample, and then bead lysing. 50 uL of soluble material saved.

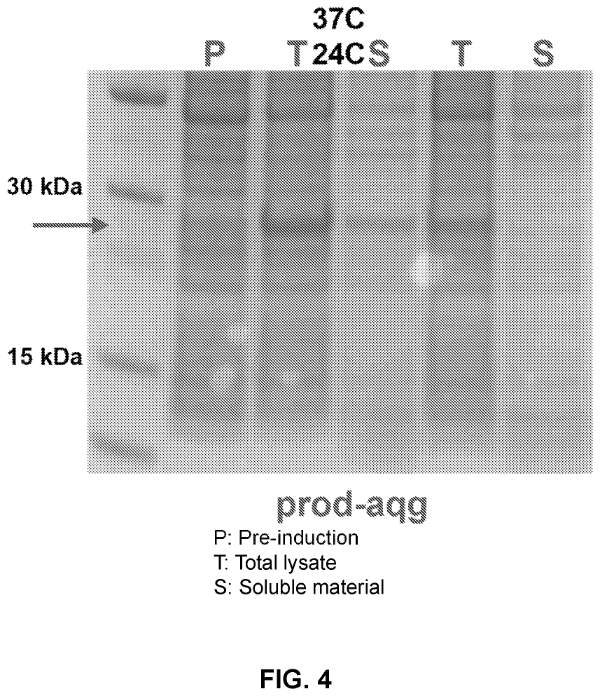

SDS-PAGE: 30 uL of samples above+20 uL 4×loading buffer. 90° C., 5 mins, loaded onto 4-20% GenScript Gel in Tris-MES buffer and ran on gel at 160V 45 mins. The results are presented in the left-most gel image in FIG. 4.

MBD fusion constructs were found to be mostly soluble simply with a 37° C. grow-up and 37° C. expression period. 2 L of each fusion protein
prod-aqg (N-terminal his tag, AviTag, MBD2_143-220, GlySer Linker, MBD2_143-220, BirA substrate peptide) 24.25 kDa, pI˜9.63
prod-bqi, (N-terminal his tag, AviTag, MBD2_143-220, GlySer Linker, BirA substrate peptide) 15.6 kDa, pI˜9.0
prod-arj (N-terminal his tag, MBD2_143-220×2) 20 kDa, pI˜9.86

30C starter cultures were grown from cell stocks generated in Example 1. The cultures underwent a 37C grow-up until an OD600 between 1.0 and 2.0. 1 mM IPTG induction. 4-5 hours expression at 37° C.

Inoculated 4×500 mL TB/Kan in 2L Erlenmeyer flasks for each. Cultures were induced at a final concentration of 1 mM IPTG and grown for a further 4 hours at 37° C., 250 rpm. Cells were pelleted 45 mins 5 krpm 4C. Cell pellets were stored at −20° C.

Cell paste processing: The 8.5 g pellet was first resuspended in 46 mL of Nickel buffer A (15 mM Tris-HCl pH 7.5, 400 mM NaCl, 20 mM imidazole, 10% glycerol). Then Lysis via sonication at 60% power; 30s on 30s off 20 cycles. 60 mL whole cell lysate was brought to 0.3% PEI by addition of 1.85 mL 9.7% PEI stock.PEI/lysate mix incubated on ice 4° C. 1 hr. Discarded PEI pellet, and brought sup to 35% AmSO Discarded 35% AmSO4 pellet and then brought to 70%. Incubated 30 mins, 4° C., and span down insoluble material. Pellet retained and stored at −20 until use.

Nickel column: Pellet was resuspended in 5×cell breakage Nickel Buffer A (50 mL). Loaded onto 3×5 mL freshly nickel charged capto chelating HiTrap columns (3 mL/min). Then a 5CV 2M salt nickel buffer wash. (3 mL/min) was performed. A 0-75% B elution over 10CV and caught 1.5 mL fractions. (3 mL/min). Ran fractions out on a Tris-MES 4-20% SDS-PAGE gel. The isolated polypeptide formed a band on the ge at 30 kDa.

Heparin column: Fractions D6 through G2 from the nickel elution were pooled (˜50 mL) and diluted to −200 mM NaCl with universal dilution buffer. The fractions were then loaded onto 3×5 mL Capto Heparin Impres Hi-Traps at 3 mL/min. They were then eluted 0-75% buffer B over 8CV.

A consequential amount of material flowed through the heparin column, and produced a small peak and one massive peak.

Running fractions on an SDS-PAGE; increasing loading dye [DTT] by 10 mM, using Tris-MOPS, and using a 4-12% gel was performed to see if the protein migrates the same way in different buffering conditions (Gel 2). 160V, 35 mins. It was found that using this column provided cleaner results and provided an isolated band representing the polypeptide at 30 kDa.

Fractions D8 through E2 (˜12 mL @ 4.5 mg/mL, −47.5 mS/cm) were pooled and mixed. 6 mL of this pool was added to 6 mL 100% glycerol. Resultant: 12 mL @−2.25 mg/mL, in −10 mM Tris-HCl pH 7.5, 250 mM NaCl, 0.1 mM EDTA, 50% glycerol.

SEC: The remaining pool was span-concentrated (Amicron 10 kDa MWCO, 1.7 mL) down from −6 mL to 500 uL and subjected to the Superdex 200 increase 15/300 size-exclusion chromatography column, 0.25 mL/min, in a 350 mM NaCl buffer. Caught 500 uL fractions.

Running on SDS-PAGE gel appeared to reveal a pure product which that oligomerized in a stable manner under these SDS conditions.

The prior sample was in −350 mM NaCl. In subsequent iterations the salt levels were increased increase

Cell paste processing: The 8.0 g pellets were first resuspended in 46 mL of Nickel buffer A (15 mM Tris-HCl pH 7.5, 400 mM NaCl, 20 mM imidazole, 10% glycerol). Lysis via sonication. 60% power; 30s on 30s off 20 cycles. 60 mL whole cell lysate was brought to 0.3% PEI by addition of 1.85 mL 9.7% PEI stock. PEI/lysate mix incubated on ice 4° C. 1 hr. Discarded PEI pellet, and brought sups (45 mL) to 70% AmSO4 with 22.39 g AmSO4. Samples were swirled often and incubated at 4° C. for 30 mins. Insoluble material pelleted 45 mins 15krpm 4° C. Pellet retained and stored at −20 until use.

Nickel column: Each pellet was resuspended in 5×cell breakage Nickel Buffer A (50 mL). Loaded onto 3×5 mL freshly nickel charged capto chelating HiTrap columns (3 mL/min). Columns were washed 1M NaOH/200 mM Tris/dH2O between runs. 5CV 2M salt nickel buffer wash. (3.2 mL/min). 0-75% B elution over 10CV. Caught 1.5 mL fractions. (3.2 mL/min). Fractions ran out on a Tris-MES 4-20% SDS-PAGE gel (Gel 4)

Both studied polypeptides were solubly expressed and stuck to nickel resin as intended.

Heparin column: elutions were pooled and loaded onto a heparin column. It was anticipated that the differing number of MBDs in the polypeptides would give a difference in affinity for the heparin column.

Pooling 750 uL from each ‘aCat177’ elution fractions E4 thru F10. Pooling 750 uL with the above pool of ‘aCat179’ elution fractions C6 thru D12. Total=28.5 mL @ 400 mM NaCl. Diluting this pool with 41.5 mL 100 mM NaCl buffer resulting in −50 mL of −222 mM NaCl load.

### Example 2

Generated several constructs for MBD2 and BirA ligase. Genetic sequences for the following fusion proteins of the invention were created: prod-aqg: N-terminal his tag, AviTag, MBD2_143-220, GlySer Linker, MBD2_143-220, BirA substrate peptide, aCat179: N-terminal his tag, AviTag, MBD2_143-220, GlySer Linker, BirA substrate peptide, prod-pqi: N-terminal his tag, MBD2_143-220, GlySer Linker, & prod-arj: BirA ligase. Placed all gene designs in pD441 vector backbone (strong RBS, T5 promoter) and pD1841 (strong RBS, phosphate starvation promoter).

As shown in FIG. 4, these fusion proteins were all successfully expressed and detected using E. coli.

>BirA ligase, prod-aqh
(SEQ ID NO: 1)
MKDNTVPLKLIALLANGEFHSGEQLGETLGMSRAAINKHIQTLRDWGVD
VFTVPGKGYSLPEPIQLLNAKQILGQLDGGSVAVLPVIDSTNQYLLDRI
GELKSGDACIAEYQQAGRGRRGRKWFSPFGANLYLSMFWRLEQGPAAAI
GLSLVIGIVMAEVLRKLGADKVRVKWPNDLYLQDRKLAGILVELTGKTG
DAAQIVIGAGINMAMRRVEESVVNQGWITLQEAGINLDRNTLAAMLIRE
LRAALELFEQEGLAPYLSRWEKLDNFINRPVKLIIGDKEIFGISRGIDK
QGALLLEQDGIIKPWMGGEISLRSAEK
>MBD2, prod-aqg
(SEQ ID NO: 2)
MHHHHHHAGGLNDIFEAQKIEWHEDTGGSATESGKRMDCPALPPGWKKE
EVIRKSGLSAGKSDVYYFSPSGKKFRSKPQLARYLGNTVDLSSFDFRTG
KMMPSKLQKDDDKEFGGGGSGGGGSGGGGATESGKRMDCPALPPGWKKE
EVIRKSGLSAGKSDVYYFSPSGKKFRSKPQLARYLGNTVDLSSFDFRTG
KMMPSKLQKLHHILDAQKMVWNHRG*
>MBD2, prod-bqi
(SEQ ID NO: 3)
MHHHHHHGAGGLNDIFEAQKIEWHEDTGGSATESGKRMDCPALPPGWKK
EEVIRKSGLSAGKSDVYYFSPSGKKFRSKPQLARYLGNTVDLSSFDFRT
GKMMPSKLQKDDDKEFGGGGSGGGGSGGGGLHHILDAQKMVWNHRG*
>MBD2_prod-arj
(SEQ ID NO: 4)
MHHHHHHATESGKRMDCPALPPGWKKEEVIRKSGLSAGKSDVYYFSPSG
KKFRSKPQLARYLGNTVDLSSFDFRTGKMMPSKLQKDDDKEFGGGGSGG
GGSGGGGATESGKRMDCPALPPGWKKEEVIRKSGLSAGKSDVYYFSPSG
KKFRSKPQLARYLGNTVDLSSFDFRTGKMMPSKLQKG*
>MDB2-Isoform-alternative-1
(SEQ ID NO: 5)
MRAHPGGGRC CPEQEEGESA AGGSGAGGDS AIEQGGQGSA
LAPSPVSGVR REGARGGGRG RGRWKQAGRG GGVCGRGRGR
GRGRGRGRGR GRGRGRPPSG GSGLGGDGGG CGGGGSGGGG
APRREPVPFP SGSAGPGPRG PRATESGKRM DCPALPPGWK
KEEVIRKSGL SAGKSDVYYF SPSGKKFRSK PQLARYLGNT
VDLSSFDFRT GKMMPSKLQK NKQRLRNDPL NQNKGKPDLN
TTLPIRQTAS IFKQPVTKVT NHPSNKVKSD PQRMNEQPRQ
LFWEKRLQGL SASDVTEQII KTMELPKGLQ GVGPGSNDET
LLSAVASALH TSSAPITGQV SAAVEKNPAV WLNTSQPLCK
AFIVTDEDIR KQEERVQQVR KKLEEALMAD ILSRAADTEE
MDIEMDSGDE A
>MDB2-Isoform-alternative-2
(SEQ ID NO: 6)
MRAHPGGGRC CPEQEEGESA AGGSGAGGDS AIEQGGQGSA
LAPSPVSGVR REGARGGGRG RGRWKQAGRG GGVCGRGRGR
GRGRGRGRGR GRGRGRPPSG GSGLGGDGGG CGGGGSGGGG
APRREPVPFP SGSAGPGPRG PRATESGKRM DCPALPPGWK
KEEVIRKSGL SAGKSDVYYF SPSGKKFRSK PQLARYLGNT
VDLSSFDFRT GKMMPSKLQK NKQRLRNDPL NQNKFVCTFL
L

What is claimed is:

1. A method for assessing a nucleic acid, the method comprising:
providing a sample comprising a methylated nucleic acid;
contacting the sample with a polypeptide comprising methyl-CpG-binding domain (MBD); and
capturing the methylated nucleic acid with the polypeptide to isolate the methylated nucleic acid.

2. The method of claim 1, wherein the nucleic acid is a cfDNA.

3. The method of claim 2, wherein the cfDNA is ctDNA.

4. The method of claim 1, wherein the polypeptide comprises a plurality of the MBDs.

5. The method of claim 1, wherein the polypeptide comprises or is attached to a capture tag.

6. The method of claim 5, wherein the polypeptide is attached to a solid surface via the capture tag.

7. The method of claim 6, wherein the method further includes a separation step to wash away non-captured nucleic acids.

8. The method of claim 6, wherein the solid surface is a bead, column, or flow cell.

9. The method of claim 8, wherein the solid surface comprises nickel and the capture tag is a His Tag.

10. The method of claim 5, wherein the capture tag comprises one or more of Protein A, Protein G, Streptavidin, Cell Surface Vimentin (CSV), Human Prostate-specific Membrane Antigen (PSMA), antibodies, antibody fragments, IgG Fc heavy chain, a viral protein, a chitin binding domain, a maltose binding protein, Protein L B′ repeats, and oligonucleotides.

10. The method of claim 1, wherein the MBD is from or derived from MBD2.

11. The method of claim 1, further comprising a separation step, optionally wherein the separation step includes capture on nickel and washing away uncaptured materials.

12. The method of claim 1, further comprising diagnosing the subject as having cancer based on identity and/or methylation status of the captured methylated nucleotide.

13. The method of claim 12, wherein the method is used to diagnose a subject as having minimal residual disease after receiving a cancer treatment.

14. The method of claim 1, further comprising expressing the polypeptide in e-coli using an expression vector.

15. The method of claim 1, wherein the methylated nucleic acids are less than about 200 bp in length.

16. The method of claim 15, wherein the methylated nucleic acids are derived from fragmented nucleic acids.

17. The method of claim 1, further comprising sequencing the captured methylated nucleic acid.

18. The method of claim 1, wherein the polypeptide includes a sequence selected from the group consisting of SEQ ID NO: 1, SEQ ID NO: 2, SEQ ID NO: 3, SEQ ID NO: 4, SEQ ID NO: 5, and SEQ ID NO: 6.

19. The method of claim 1, wherein the polypeptide includes a sequence selected from the group consisting of SEQ ID NO: 2, SEQ ID NO: 3, and SEQ ID NO: 4.

20. The method of claim 1, wherein the polypeptide includes a SEQ ID NO: 2 or SEQ ID NO: 3.


SEQUENCE LISTING (6 sequences)

SEQ ID NO: 1 - protein, 321 aa, synthetic construct
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 2 - protein, 221 aa, synthetic construct
MHHHHHHAGGLNDIFEAQKIEWHEDTGGSATESGKRMDCPALPPGWKKEEVIRKSGLSAGKSDVYYFSPSGKKFRSKPQLARYLGNTVDLSSFDFRTGKMMPSKLQKDDDKEFGGGGSGGGGSGGGGATESGKRMDCPALPPGWKKEEVIRKSGLSAGKSDVYYFSPSGKKFRSKPQLARYLGNTVDLSSFDFRTGKMMPSKLQKLHHILDAQKMVWNHRG

SEQ ID NO: 3 - protein, 144 aa, synthetic construct
MHHHHHHGAGGLNDIFEAQKIEWHEDTGGSATESGKRMDCPALPPGWKKEEVIRKSGLSAGKSDVYYFSPSGKKFRSKPQLARYLGNTVDLSSFDFRTGKMMPSKLQKDDDKEFGGGGSGGGGSGGGGLHHILDAQKMVWNHRG

SEQ ID NO: 4 - protein, 184 aa, synthetic construct
MHHHHHHATESGKRMDCPALPPGWKKEEVIRKSGLSAGKSDVYYFSPSGKKFRSKPQLARYLGNTVDLSSFDFRTGKMMPSKLQKDDDKEFGGGGSGGGGSGGGGATESGKRMDCPALPPGWKKEEVIRKSGLSAGKSDVYYFSPSGKKFRSKPQLARYLGNTVDLSSFDFRTGKMMPSKLQKG

SEQ ID NO: 5 - protein, 411 aa, synthetic construct
MRAHPGGGRCCPEQEEGESAAGGSGAGGDSAIEQGGQGSALAPSPVSGVRREGARGGGRGRGRWKQAGRGGGVCGRGRGRGRGRGRGRGRGRGRGRPPSGGSGLGGDGGGCGGGGSGGGGAPRREPVPFPSGSAGPGPRGPRATESGKRMDCPALPPGWKKEEVIRKSGLSAGKSDVYYFSPSGKKFRSKPQLARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEMDIEMDSGDEA

SEQ ID NO: 6 - protein, 241 aa, synthetic construct
MRAHPGGGRCCPEQEEGESAAGGSGAGGDSAIEQGGQGSALAPSPVSGVRREGARGGGRGRGRWKQAGRGGGVCGRGRGRGRGRGRGRGRGRGRGRPPSGGSGLGGDGGGCGGGGSGGGGAPRREPVPFPSGSAGPGPRGPRATESGKRMDCPALPPGWKKEEVIRKSGLSAGKSDVYYFSPSGKKFRSKPQLARYLGNTVDLSSFDFRTGKMMPSKLQKNKQRLRNDPLNQNKFVCTFLL